(19) United States
(12) Patent Application Publication
(10) Pub. No.: US 2005/0246025 A1
(43) Pub. Date: Nov. 3, 2005

(54) Fracture repair system utilizing custom modulus

(76) Inventor: Richard F. Kyle, Long Lake, MN (US)

Correspondence Address: KAGAN BINDER, PLLC, SUITE 200, MAPLE ISLAND BUILDING, 221 MAIN STREET NORTH, STILLWATER, MN, 55082, US

(21) Appl. No.: 11/110,974

(22) Filed: Apr. 19, 2005

Related U.S. Application Data
(60) Provisional application No. 60/563,322, filed on Apr. 19, 2004.

(51) Int. Cl. A61F 2/08

(52) U.S. Cl. 623/18.11; 623/14.12

(57) ABSTRACT

A system and method for repairing fractures to articular joint components while maintaining more natural tissue than with other devices. The system and method utilizes materials having at least one portion with a modulus customized for preservation of natural articular cartilage it contacts, according to various features of the patient.

### FIELD OF THE INVENTION

The field of the invention relates to a medical prosthesis utilizing modulus matching principles.

### BACKGROUND OF THE INVENTION

Within the field of orthopedics, there are numerous examples of prostheses made from a wide range of materials. In the past, implantable device standards for load bearing joints required metallic components to ensure strength and availability. With improvements in materials science new devices were made of various types of non-metallic materials, including plastics, or composites having desired biocompatibility and stress-resistant features. In the field of synovial joint repair a standard evolved which urged complete replacement of all articular surfaces. This is still common in the surgical repair of hip fractures, for example, where a metallic ball is designed for femoral head replacement and interaction with an artificial acetabular cup, thus replacing natural tissue and bone on both sides of the joint. These artificial components may be quite expensive. However, it is well known that certain failure rates over time exist, the reduction of which are the subjects of the inventions herein.

### SUMMARY OF THE INVENTION

A system and method for repairing fractures to articular joint components while maintaining more natural tissue than with other devices. The system and method utilizes materials having at least one portion with a modulus customized for preservation of natural articular cartilage it contacts, according to various features of the patient.

### DESCRIPTION OF THE DRAWINGS

FIG. 1 is a side elevation view of a total hip replacement device.

FIG. 2 is a side elevation view of a natural hip joint, representative of other articular joints.

FIG. 3 is a side elevation view of a fractured femoral head portion of a human.

FIG. 4 is a side elevation view of an artificial femoral head assembly in place.

### DETAILED DESCRIPTION OF THE INVENTION

The fracture repair system of this invention is designed to reduce the damage to natural healthy tissue during repair of part of a joint injured by trauma or, in certain cases, disease. It is recognized that those patients having damage on both sides of a joint, e.g. a femoral head and the acetabulum, are likely more suited to receive a complete or total joint repair. In that case a replacement system is often comprised of matching artificial components, such as a metallic or ceramic femoral head 10 for use in contact with an acetabular cup 13 made of plastic, metal or other hard material, as shown in FIG. 1. Known problems with such procedures and implanted systems include loss of considerable natural tissue, undesired material degradation over time, as well as micro-particulate generation into the joint by the forces of the two materials typically during loaded movement.

Certain joints such as fingers, toes and wrists have been repaired with hemi-arthroplasty techniques to repair one side of the joint. The materials selected for the prostheses include only the known hardened materials as in the total replacement procedures, and with little other critical design parameters involved in such selection. Such selection processes have led to continued problems similar to those noted above in relation to total replacements surgeries. Applicants have identified improved selection techniques and critical modulus matching device structures for maximizing retention of natural tissue during joint repairs and optimizing the regenerative processes of associated articular cartilage. These devices and techniques facilitate or accelerate healing and likely minimize secondary vulnerability to tissue degeneration or disease processes later. Another aspect of these discoveries include accommodating the different tissue structures of different age or gender or injury groups to further prevent dramatic surface irregularities between the natural articular cartilage and the implanted opposing artificial surface, whether directly or indirectly such as when such artificial surface has a coating applied to it.

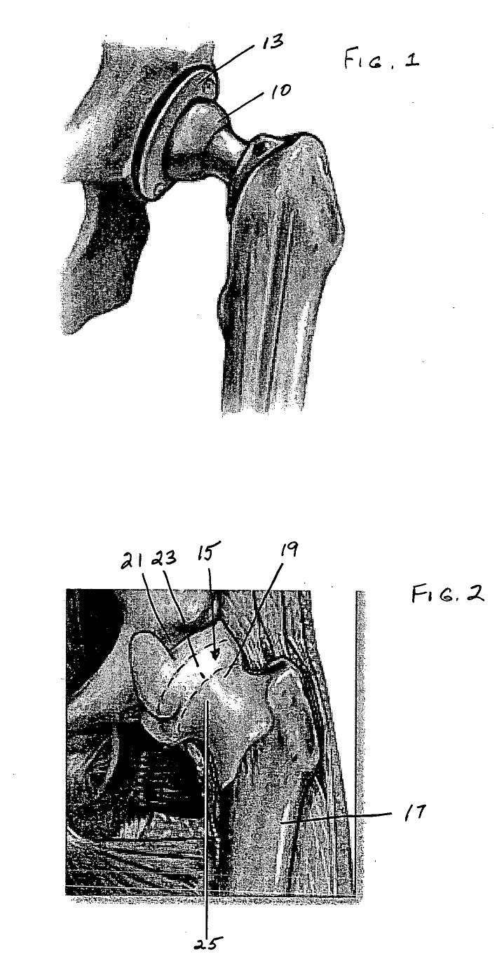

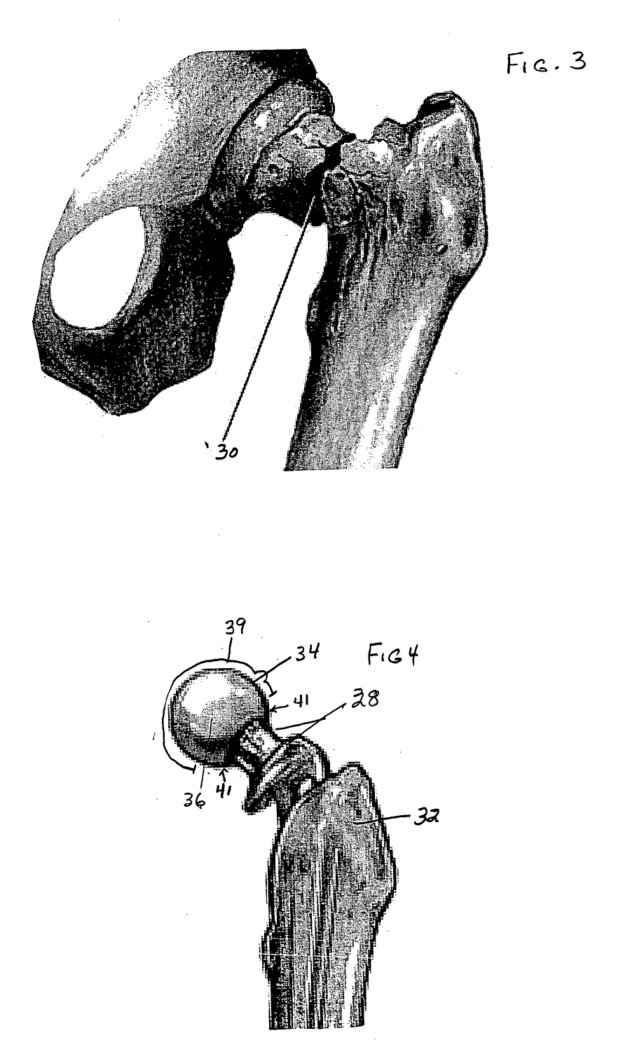

FIG. 2 illustrates one example of a natural articular joint 15. In this example the femur 17 is shown with an intact healthy femoral head 19 in normal placement within the acetabulum 21. Natural articular cartilage 23 is shown in place performing properly. The outer synovial membrane 25 encases this joint and retains the synovial fluid exuded during the cartilage loading process. FIG. 3 illustrates a fractured femoral head portion 30 associated with an otherwise healthy structure. For ease of illustration the synovial membrane is not shown in this figure. Here, as with other similar articular joints, it is desirable to retain as much natural tissue as possible during any repair.

Accordingly, FIG. 4 illustrates the surgically corrected proximal femur 32 with an artificial femoral head 34 constructed of a material having a modulus more similar to that of the natural cartilage than is metal or other hard materials. In one embodiment this femoral head comprises a polyurethane or similar material 36 with an accommodated modulus suitable for providing improved compatibility with the adjacent articular cartilage which is at the opposing acetabular region. The head 14 accommodated modulus material is designed to fit onto a standard femoral stem 38 with a trunion or moris taper, or other suitable connection structure. The material of head 34 is designed so that the portion 39 contacting the retained natural articular cartilage is softer than the conventional steel head, and the portion 41 connecting to the trunion or taper has suitable strength and hardness for its purpose, i.e. one or more of the locations on the head may have material(s) of differing hardness or other feature relating to force on the adjacent natural tissue. Such locations may be radial placed or along the surface. This important feature will preserve the articular cartilage on the acetabular side of the joint and will promote longevity of the repaired joint. This results in less morbidity, greater mobility, and improved pain management. Moreover, this technique of modulus matching and tissue retention enables longer effective life of the implanted and natural surfaces of the joint. Cost and manufacturing advantages may also result.

The mechanical response of cartilage is related to the flow of fluid through the tissue. When deformed, the fluid flows through the cartilage and across the articular surface. Thus, modeling of cartilage accounts for both the interstitial fluid and the solid components. i.e., proteoglycans, collagen, cells, and lipids. Applicants have used the various modeling techniques to determine tissue criteria such as aggregrate modulus and permeability values for specific patient classes based on age, gender, stress-strain history, and other markers. For example, the higher the aggregate modulus, the less the tissue deforms under a given load. The aggregate modulus of cartilage is normally in the range of 0.5 to 0.9 MPa. By further calculation using Poisson's ratio, compression tests and Darcy's law, the estimated Young's modulus for cartilage is in the range of 0.45 to 0.80 MPa. Together, this information aids in formulating the optimum modulus for material 36 to allow a cushioning effect under load and to ease the shear forces, and a relative hydrophilicity and wetability to maintain microelastodynamic lubrication (i.e. boundary lubrication at low loads and fluid film lubrication at high loads). Due to the relatively low coefficient of friction of normal synovial joints of about 0.001, the improvements of the invention result in orders of magnitude improvement over other materials having less critical design criteria. Matching of the modulus further includes recognition of the link between mechanical stimulation of the joint and production and activity of chondrocytes. This and other factors (such as degree of collagen fiber stiffness and proteoglycan status) aid the surgeon in providing the optimum material choice for each specific patient.

It is known that the failure rate of artificial or even autologous cartilage repair varies due to many factors. Moreover, damage to cartilage likely results in disruption of normal processes which enable load carrying ability of the tissue and the associated lubrication processes. Certain analyses also demonstrate that loading of damaged cartilage leads to reduction in remodeling capacity and a predisposition to degeneration, undesired chondrocyte production, and osteoarthritis. In contrast, careful pre-operative analysis, marker identification, and even indentation probe stiffness and impaction analysis during surgery provides valuable data with which to determine the optimum prosthesis material for interaction with the natural tissue, either bone or cartilage. Further quantitative measurements such as relative balance of oxidant/antioxidant factors may aid in proper material or coatings selection. By careful selection of material 36 and retention of all the natural cartilage and other tissue possible, substantial patient care improvements are now possible.

While certain embodiments of the invention have been shown in greater detail than others, such detail is intended to apply as well to alternate embodiments of the inventions described herein without limiting the scope of the disclosure.

1. A prosthetic device for use in an articular joint repair system having an articular tissue contacting component comprising a material with a custom modulus designed to substantially match the desired value of modulus for the specific patient.

2. The component of claim 1 in which the material is a polymer.

3. The material of claim 3 in which the polymer has at least two portions having different material values of modulus.

4. A method of selecting a custom modulus component for use in a prosthesis designed for an articular joint repair comprising the steps of:
a. identifying the patient-specific tissue criteria of the patient's natural articular cartilage; and
b. identifying a material for use with the prostheses portion in contact with the natural articular cartilage that is best customized to the patient-specific tissue criteria.
